(19) United States
(12) Patent Application Publication
(10) Pub. No.: US 2024/0148812 A1
(43) Pub. Date: May 9, 2024

(54) PHARMACEUTICAL COMPOSITION FOR PREVENTING OR TREATING DISEASES CAUSED BY VASCULAR AGING COMPRISING SALVIA MILTIORRHIZA EXTRACT OR PAEONIA LACTIFLORA EXTRACT AS AN ACTIVE INGREDIENT

(71) Applicant: USCAREPHARM CO.,LTD, Suwon-si (KR)

(72) Inventors: Sukjin SONG, Yongin-si (KR); Juah SON, Yongin-si (KR); Mi-Ran CHA, Suwon-si (KR); Byulnim OH, Seoul (KR); Sangwoo BANG, Seongnam-si (KR); Jinwook CHA, Hwaseong-si (KR)

(73) Assignee: USCAREPHARM CO.,LTD, Suwon-si (KR)

(21) Appl. No.: 18/386,261

(22) Filed: Nov. 2, 2023

(30) Foreign Application Priority Data
Nov. 3, 2022 (KR) 10-2022-0145304

(51) Int. Cl. A61K 36/537; A61K 36/65; A61P 9/14

(52) U.S. Cl. CPC A61K 36/537; A61K 36/65; A61P 9/14; A61K 2236/33; A61K 2236/331; A61K 2236/333

(57) ABSTRACT

The present disclosure relates to a pharmaceutical composition for preventing or treating diseases caused by vascular aging, including a Salvia miltiorrhiza extract or a Paeonia lactiflora extract as an active ingredient, wherein it was found that a single extract of each of Salvia miltiorrhiza and Paeonia lactiflora or a mixed extract of both mixed in a specific weight ratio showed effects of improving symptoms by vascular aging such as cytoprotection against cytotoxicity due to vascular aging, promotion of nitric oxide production, inhibition of collagen fiber production, suppression of vascular thickness increase, and inhibitory activities on fibrosis by collagen deposition, thereby being useful as a composition for preventing, treating, or ameliorating diseases or symptoms caused by vascular aging.

### CROSS-REFERENCE TO RELATED APPLICATION

This application claims the benefit of Korean Patent Application No. 10-2022-0145304 filed on Nov. 3, 2022, in the Korean Intellectual Property Office, the entire disclosure of which is incorporated herein by reference for all purposes.

### BACKGROUND

### 1. Field of the Invention

The present disclosure relates to a pharmaceutical composition for preventing or treating diseases caused by vascular aging, including a Salvia miltiorrhiza extract or a Paeonia lactiflora extract as an active ingredient.

### 2. Description of the Related Art

Cell aging, a phenomenon in which normal somatic cells may no longer divide after dividing a certain number of times, contributes to aging of individuals and tissues and is an important mechanism to inhibit abnormal proliferation of cells and cancer formation. Cellular aging is induced by shortening of telomeres, the ends of chromosomes, due to repeated division of somatic cells, increased activity of oncogenes or cancer suppressor genes, excessive oxidative stress, ultraviolet rays, irradiation, cytotoxic substances such as anticancer agents, and inflammatory responses, causing various diseases such as rheumatoid arthritis, osteoarthritis, inflammatory diseases, and cancer.

Vascular endothelial cells are a layer of cells that make up the lumen of blood vessels and play a major regulatory role in maintaining vascular homeostasis, such as vasodilation and vasoconstriction, proliferation and migration of vascular smooth muscle, and thrombogenesis and thrombolysis. Dysfunction of vascular endothelial cells may disrupt the balance of vascular homeostasis to cause various diseases, leading to vascular endothelial cell dysfunction due to cellular aging.

Aged vascular endothelial cells show reduced blood vessel contractility due to reduced nitric oxide (NO), endothelial nitric oxide synthase (eNOS) expression, and prostacyclin production, facilitated thrombogenesis due to increased expression of type I plasminogen activator (PAI-1), and narrowed blood vessels due to inflammatory responses as expression of inter-cellular adhesion molecule 1 (ICAM-1), interleukin-1 (IL-1), and IL-8 is increased, symptoms of which lead to various vascular diseases such as arteriosclerosis, hyperlipidemia, angina pectoris, myocardial infarction, and stroke.

As such, the aging of cells including vascular endothelial cells may cause various diseases, such that research is being actively conducted to improve symptoms of cell aging.

### SUMMARY

### Problem to be Solved by the Invention

An object of the present disclosure is to provide a pharmaceutical composition for preventing or treating diseases caused by vascular aging, including a Salvia miltiorrhiza extract; a Paeonia lactiflora extract; or a mixed extract of Salvia miltiorrhiza and Paeonia lactiflora as an active ingredient.

Another object of the present disclosure is to provide a health functional food composition for preventing or ameliorating diseases caused by vascular aging, including a Salvia miltiorrhiza extract; a Paeonia lactiflora extract; or a mixed extract of Salvia miltiorrhiza and Paeonia lactiflora as an active ingredient.

Another object of the present disclosure is to provide a food composition for preventing or ameliorating symptoms caused by vascular aging, including a Salvia miltiorrhiza extract; a Paeonia lactiflora extract; or a mixed extract of Salvia miltiorrhiza and Paeonia lactiflora as an active ingredient.

### Means for Solving the Problem

In order to achieve the above object, the present disclosure provides a pharmaceutical composition for preventing or treating diseases caused by vascular aging, including a Salvia miltiorrhiza extract; a Paeonia lactiflora extract; or a mixed extract of Salvia miltiorrhiza and Paeonia lactiflora as an active ingredient.

In addition, the present disclosure provides a health functional food composition for preventing or ameliorating diseases caused by vascular aging, including a Salvia miltiorrhiza extract; a Paeonia lactiflora extract; or a mixed extract of Salvia miltiorrhiza and Paeonia lactiflora as an active ingredient.

In addition, the present disclosure provides a food composition for preventing or ameliorating symptoms caused by vascular aging, including a Salvia miltiorrhiza extract; a Paeonia lactiflora extract; or a mixed extract of Salvia miltiorrhiza and Paeonia lactiflora as an active ingredient.

### Effects of the Invention

According to the present disclosure, it was found that a single extract of each of Salvia miltiorrhiza and Paeonia lactiflora or a mixed extract of both mixed in a specific weight ratio showed effects of improving symptoms by vascular aging such as cytoprotection against cytotoxicity due to vascular aging, promotion of nitric oxide production, inhibition of collagen fiber production, suppression of vascular thickness increase, and inhibitory activities on fibrosis by collagen deposition, thereby being useful as a composition for preventing, treating, or ameliorating diseases or symptoms caused by vascular aging.

### BRIEF DESCRIPTION OF THE DRAWINGS

FIG. 1 is graphs showing results of analyzing effects of a mixed extract of Salvia miltiorrhiza and Paeonia lactiflora on blood pressure, intima-media thickness, and vascular collagen fibrosis using a vascular aging-induced animal model. In the case of blood pressure, intima-media thickness, and collagen fibrosis, vascular aging-induced G2 was statistically significantly increased compared to normal group G1, meaning that vascular aging occurred. For systolic blood pressure, compared with G2, G5 and G6 had a valid difference of p<0.05 and p<0.01 levels, respectively, and in case of G6, diastolic blood pressure had a valid difference of p<0.01, while intima-media thickness and vascular collagen fibrosis had valid differences of p<0.05 and p<0.01 levels in G5 and G6, respectively.

FIG. 2 shows microscopic observation results of effects of a mixed extract of Salvia miltiorrhiza and Paeonia lactiflora on intima-media thickness and vascular collagen fibrosis by staining aorta of vascular aging-induced animals.

### DETAILED DESCRIPTION

Hereinafter, the present disclosure will be described in more detail.

In addition, the present disclosure provides a pharmaceutical composition for preventing or treating diseases caused by vascular aging, including a Salvia miltiorrhiza extract; a Paeonia lactiflora extract; or a mixed extract of Salvia miltiorrhiza and Paeonia lactiflora as an active ingredient.

The extract may be extracted with water, a low-grade C1-C4 alcohol, or a mixed solvent thereof, preferably with a 60-80% (v/v) aqueous ethanol solution, and more preferably with a 70% (v/v) aqueous ethanol solution.

The mixed extract may be obtained by mixing the Salvia miltiorrhiza extract and the Paeonia lactiflora extract in a weight ratio of 1:0.1 to 10, but is not limited thereto.

In addition, the extract may exhibit a cytoprotective effect against cytotoxicity induced by vascular aging, promote nitric oxide production, and inhibit collagen fiber production.

The disease may be one or more selected from the group consisting of, but is not limited to, atherosclerosis, hyperlipidemia, hypertension, angina, myocardial infarction, stroke, cardiac arrhythmia, heart failure, endocarditis, ischemic cerebrovascular disease, and ischemic heart disease.

As used herein, the term “Salvia miltiorrhiza” refers to a perennial herbaceous plant belonging to the Labiatae family, with a shape resembling Ginseng in red color to be known as red sage. The dried root of Salvia miltiorrhiza has traditionally been used to improve blood circulation and treat congestion and is also called Geukseoncho, Mokyangyu, Bunmacho, red sage, and red root.

As used herein, the term “Paeonia lactiflora ” refers to a cold-resistant perennial herb belonging to the Ranunculaceae family that grows in the low mountains of the central and northern regions. From old times, it has been cultivated for ornamental and medicinal purposes while the root is used as a medicine in Chinese medicine, wherein white peony root was used as anemia treatment and analgesics, and red peony root as antihypertensives and antipyretics.

The pharmaceutical composition of the present disclosure may be prepared in a unit dose form or prepared by infusion in a multi-dose container through formulation using pharmaceutically acceptable carriers according to a method that may be easily carried out by a person skilled in the art to which the present disclosure pertains.

The pharmaceutically acceptable carriers are those commonly used in preparation, including lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and, mineral oil, but are not limited thereto. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional formulation.

The pharmaceutical composition may be formulated in the form of one or more skin external preparations selected from the group consisting of injectable formulations such as aqueous solutions, suspensions, and emulsions, pills, capsules, granules, tablets, creams, gels, patches, sprays, ointments, emplastrum agents, lotions, liniments, pastas, and cataplasmas, but are not limited thereto.

The pharmaceutical composition may include a pharmaceutically acceptable carrier and a diluent, which are additional for the formulation. The pharmaceutically acceptable carrier and diluent include excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol, but are not limited thereto. The pharmaceutically acceptable carrier and diluent may be biologically and physiologically compatible with subjects. Examples of the diluent may include saline, aqueous buffers, solvents, and/or dispersion media, but are not limited thereto.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, powder preparation, granules, emulsions, hard capsules, soft capsules, syrups, and elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the degree of diseases, administration duration of the composition, administration methods, administration periods or intervals, excretion rate, and drug forms, and be appropriately selected by those skilled in the art. For example, the dosage may be in the range of about 0.1 to 10,000 mg/kg but is not limited thereto, while it may be administrated in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on formulation methods, administration methods, administration duration, and administration routes of the pharmaceutical composition, and those skilled in the art may easily determine and prescribe the dosage effective for desired treatment. Administration of the pharmaceutical composition of the present disclosure may be conducted once a day or several times in divided doses.

In addition, the present disclosure provides a health functional food composition for preventing or ameliorating diseases caused by vascular aging, including a Salvia miltiorrhiza extract; a Paeonia lactiflora extract; or a mixed extract of Salvia miltiorrhiza and Paeonia lactiflora as an active ingredient.

The present disclosure may be generally used as a commonly used food.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to regulate nutrients for the structure and function of the human body or derive effectiveness in health care such as physiological actions.

The health functional food composition may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemical compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action to suppress or delay a disease or symptom caused by vascular aging by administration of the composition according to the present disclosure.

The term “treatment” as used herein refers to any action to improve or beneficially modify a disease caused by vascular aging by administration of the composition according to the present disclosure.

The term “amelioration” as used herein refers to any action to improve a bad state of a disease or symptom caused by vascular aging by administration of the composition according to the present disclosure.

In addition, the present disclosure provides a food composition or health functional food composition for preventing or ameliorating symptoms caused by vascular aging, including a Salvia miltiorrhiza extract; a Paeonia lactiflora extract; or a mixed extract of Salvia miltiorrhiza and Paeonia lactiflora as an active ingredient.

The symptoms induced by vascular aging may be one or more selected from the group consisting of a decrease in the elasticity of blood vessels, an increase in the stiffness of blood vessels, expansion of an inner diameter of blood vessels, an increase in the thickness of the vascular wall of blood vessels, degeneration of the intima-media membrane of blood vessels, a decrease in dilatability of blood vessels, insufficiency of endothelial cells of blood vessels, an increase in reactive oxygen species (ROS) of blood vessels, an inflammatory response in blood vessels, proliferation of smooth muscle cells in the subendothelial space of blood vessels, a decrease in blood flow rate in blood vessels, and an increase in blood pressure, but are not limited thereto.

Hereinafter, in order to help the understanding of the present disclosure, example embodiments will be described in detail. However, the following example embodiments are only illustrative of contents of the present disclosure, and the scope of the present disclosure is not limited to the following example embodiments. Example embodiments of the present disclosure are provided for more complete explanation of the present disclosure to a person of average skill in the art.

### [Preparation Example 1] Preparation of Extracts

Extracts were prepared under a single or mixed extraction condition for each of Salvia miltiorrhiza and Paeonia lactiflora; and an alcohol (ethanol) concentration (0, 30, 70 and 100%) condition. First, as shown in Table 1, Salvia miltiorrhiza and Paeonia lactiflora were mixed according to each condition by weight ratio, and then water or 30 to 100% alcohol was selected as an extraction solvent according to each condition, followed by extraction. The temperature of the extract solution was maintained at 70° C. using a multi-heating mantle (WiseTherm), and a reflux cooling device was connected to a low-temperature constant temperature water bath (A&D) to apply the reflux-cooled extract to water bath at 45° C. and concentration under reduced pressure less than or equal to 200 mbar by utilizing a vacuum rotation concentrator (EYELA) so as to finally obtain extracts for each condition. Thereafter, the yield (%) of the extracts according to each of the above conditions was determined.

TABLE 1

| Preparation | | | Extraction |
|---|---|---|---|
| Example | Salvia miltiorrhiza | Paeonia lactiflora | solvent |
| 1 | 1 | 0 | Water |
| 2 | 0 | 1 | Water |
| 3 | 1 | 1 | Water |
| 4 | 2 | 1 | Water |
| 5 | 4 | 1 | Water |
| 6 | 6 | 1 | Water |
| 7 | 1 | 2 | Water |
| 8 | 1 | 4 | Water |
| 9 | 1 | 6 | Water |
| 10 | 1 | 0 | 30% Ethanol |
| 11 | 0 | 1 | 30% Ethanol |
| 12 | 1 | 1 | 30% Ethanol |
| 13 | 2 | 1 | 30% Ethanol |
| 14 | 4 | 1 | 30% Ethanol |
| 15 | 6 | 1 | 30% Ethanol |
| 16 | 1 | 2 | 30% Ethanol |
| 17 | 1 | 4 | 30% Ethanol |
| 18 | 1 | 6 | 30% Ethanol |
| 19 | 1 | 0 | 70% Ethanol |
| 20 | 0 | 1 | 70% Ethanol |
| 21 | 1 | 1 | 70% Ethanol |
| 22 | 2 | 1 | 70% Ethanol |
| 23 | 4 | 1 | 70% Ethanol |
| 24 | 6 | 1 | 70% Ethanol |
| 25 | 1 | 2 | 70% Ethanol |
| 26 | 1 | 4 | 70% Ethanol |
| 27 | 1 | 6 | 70% Ethanol |
| 28 | 1 | 0 | 100% Ethanol |
| 29 | 0 | 1 | 100% Ethanol |
| 30 | 1 | 1 | 100% Ethanol |
| 31 | 2 | 1 | 100% Ethanol |
| 32 | 4 | 1 | 100% Ethanol |
| 33 | 6 | 1 | 100% Ethanol |
| 34 | 1 | 2 | 100% Ethanol |
| 35 | 1 | 4 | 100% Ethanol |
| 36 | 1 | 6 | 100% Ethanol |

### [Experimental Example 1] Cell Culture

1-1. Culture of Human Aortic Endothelial Cells

Human aortic endothelial cells (hereinafter referred to as HaoEC)(PromoCell®) were cultured in Endothelial Cell Growth Medium MV and maintained at 37° C. with 5% CO2 in a incubator. When cells reached about 80% confluency, the cells were isolated and subcultured using a DetachKit.

1-2. Culture of Human Aortic Smooth Muscle Cells

Human aortic smooth muscle cells (hereinafter referred to as HAoSMC, PromoCell®) were cultured in Smooth Muscle Cell Growth Medium 2 and maintained at 37° C. with 5% CO2 in a incubator. When cells reached about 80% confluency, the cells were isolated and subcultured using a DetachKit.

### [Example 1] Analysis of Cytotoxicity

1-1. Analysis of Cytotoxicity by Oxidative Stress

In order to determine whether the Salvia miltiorrhiza or Paeonia lactiflora extract exhibits a cytoprotective effect during vascular aging caused by oxidative stress, HAoECs were treated treated with hydrogen peroxide (hereinafter referred to as H₂O₂) to induce vascular aging by oxidative stress, and cytotoxicity was analyzed. Specifically, HAoECs were seeded in a 96 well plate at 5×10⁴ cells/well and then incubated for 24 hours in an incubator at 37° C. under 5% CO₂. The cells were treated with 50 μg/ml of the extracts in Preparation Examples 1 to 36 and H₂O₂ and incubated for 48 hours. 10 μL of Quanti-Max™ WST-8 Cell Viability Assay Kit solution was dispensed based on a 200 μL medium, and cytotoxicity was analyzed using a cell counting kit (CCK) at absorbance 450 nm after 1 hour. Cytoprotective ability against cytotoxicity was calculated using the following Equation 1.

Cytoprotective ability (%)={(Absorbance of untreated group−Absorbance of extract-treated group)/(Absorbance of untreated group−Absorbance of H₂O₂-treated group)}×100 [Equation 1]

As a result, as shown in Table 2, Cell viability was reduced at the H₂O₂-treated group due to cytotoxicity, while in Preparation Examples 1 to 36, the viability of 115% or more was restored compared to the decrease in cell viability due to H₂O₂. Specifically, 1) mixed extracts rather than single extracts and 2) ethanol extracts rather than water extracts showed superior cytoprotective ability against cytotoxicity, cytoprotective ability of the 70% ethanol extract was the best among the extraction solvents, and Preparation Example 21 showed the highest cytoprotective ability of 42.2%.

TABLE 2

| Preparation | Salvia | Paeonia | Extraction | Cytoprotective |
|---|---|---|---|---|
| Example | miltiorrhiza | lactiflora | solvent | ability (%) |
| 1 | 1 | 0 | Water | 17.1 |
| 2 | 0 | 1 | Water | 16.8 |
| 3 | 1 | 1 | Water | 24.5 |
| 4 | 2 | 1 | Water | 20.1 |
| 5 | 4 | 1 | Water | 18.7 |
| 6 | 6 | 1 | Water | 18.5 |
| 7 | 1 | 2 | Water | 20.5 |
| 8 | 1 | 4 | Water | 18.9 |
| 9 | 1 | 6 | Water | 18.7 |
| 10 | 1 | 0 | 30% Ethanol | 20.1 |
| 11 | 0 | 1 | 30% Ethanol | 19.8 |
| 12 | 1 | 1 | 30% Ethanol | 31.5 |
| 13 | 2 | 1 | 30% Ethanol | 25.7 |
| 14 | 4 | 1 | 30% Ethanol | 24.8 |
| 15 | 6 | 1 | 30% Ethanol | 20.5 |
| 16 | 1 | 2 | 30% Ethanol | 24.4 |
| 17 | 1 | 4 | 30% Ethanol | 22.5 |
| 18 | 1 | 6 | 30% Ethanol | 21.5 |
| 19 | 1 | 0 | 70% Ethanol | 28.1 |
| 20 | 0 | 1 | 70% Ethanol | 27.4 |
| 21 | 1 | 1 | 70% Ethanol | 42.2 |
| 22 | 2 | 1 | 70% Ethanol | 34.8 |
| 23 | 4 | 1 | 70% Ethanol | 31.7 |
| 24 | 6 | 1 | 70% Ethanol | 30.2 |
| 25 | 1 | 2 | 70% Ethanol | 30.8 |
| 26 | 1 | 4 | 70% Ethanol | 30.4 |
| 27 | 1 | 6 | 70% Ethanol | 30.1 |
| 28 | 1 | 0 | 100% Ethanol | 21.5 |
| 29 | 0 | 1 | 100% Ethanol | 20.8 |
| 30 | 1 | 1 | 100% Ethanol | 28.5 |
| 31 | 2 | 1 | 100% Ethanol | 26.7 |
| 32 | 4 | 1 | 100% Ethanol | 24.5 |
| 33 | 6 | 1 | 100% Ethanol | 22.6 |
| 34 | 1 | 2 | 100% Ethanol | 25.4 |
| 35 | 1 | 4 | 100% Ethanol | 22.8 |
| 36 | 1 | 6 | 100% Ethanol | 22.1 |

1-2. Analysis of Cytotoxicity by Inflammatory Responses

In order to determine whether the Salvia miltiorrhiza or Paeonia lactiflora extract exhibits a cytoprotective effect during vascular aging caused by inflammatory response, HAoEC was treated with tumor necrosis factor-α (TNF-α) to induce vascular aging by inflammatory response, and then cytotoxicity was analyzed. Specifically, HAoECs were seeded in a 96 well plate at 5×10⁴ cells/well and then incubated for 24 hours in an incubator at 37° C. under 5% CO₂. The cells were treated with 50 μg/ml of the extracts in Preparation Examples 1 to 36 and TNF-α and incubated for 48 hours. 10 μL of Quanti-Max™ WST-8 Cell Viability Assay Kit solution was dispensed based on a 200 μL medium, and cytotoxicity was analyzed using CCK at absorbance 450 nm after 1 hour. The cytoprotective ability against cytotoxicity was calculated using the following Equation 2.

Cytoprotective ability (%)={(Absorbance of untreated group−Absorbance of extract-treated group)/(Absorbance of untreated group−Absorbance of TNF-α-treated group)}×100 [Equation 2]

As a result, as shown in Table 3, Cell viability was decreased at the TNF-α-treated group, while in Preparation Examples 1 to 36, the viability was restored to some extent for the decrease in cell viability due to TNF-α. Specifically, 1) mixed extracts rather than single extracts and 2) ethanol extracts rather than water extracts showed superior cytoprotective ability against cytotoxicity, cytoprotective ability of 70% ethanol extract was the best among the extraction solvents, and Preparation Example 21 showed the highest cytoprotective ability of 38.4%

TABLE 3

| Preparation | Salvia | Paeonia | Extraction | Cytoprotective |
|---|---|---|---|---|
| Example | miltiorrhiza | lactiflora | solvent | ability (%) |
| 1 | 1 | 0 | Water | 12.5 |
| 2 | 0 | 1 | Water | 15.1 |
| 3 | 1 | 1 | Water | 25.1 |
| 4 | 2 | 1 | Water | 17.6 |
| 5 | 4 | 1 | Water | 17.1 |
| 6 | 6 | 1 | Water | 16.8 |
| 7 | 1 | 2 | Water | 17.9 |
| 8 | 1 | 4 | Water | 17.5 |
| 9 | 1 | 6 | Water | 17.0 |
| 10 | 1 | 0 | 30% Ethanol | 18.7 |
| 11 | 0 | 1 | 30% Ethanol | 22.1 |
| 12 | 1 | 1 | 30% Ethanol | 28.4 |
| 13 | 2 | 1 | 30% Ethanol | 24.2 |
| 14 | 4 | 1 | 30% Ethanol | 23.2 |
| 15 | 6 | 1 | 30% Ethanol | 23.0 |
| 16 | 1 | 2 | 30% Ethanol | 24.9 |
| 17 | 1 | 4 | 30% Ethanol | 24.7 |
| 18 | 1 | 6 | 30% Ethanol | 24.2 |
| 19 | 1 | 0 | 70% Ethanol | 22.5 |
| 20 | 0 | 1 | 70% Ethanol | 25.1 |
| 21 | 1 | 1 | 70% Ethanol | 38.4 |
| 22 | 2 | 1 | 70% Ethanol | 29.7 |
| 23 | 4 | 1 | 70% Ethanol | 27.5 |
| 24 | 6 | 1 | 70% Ethanol | 26.9 |
| 25 | 1 | 2 | 70% Ethanol | 28.9 |
| 26 | 1 | 4 | 70% Ethanol | 27.4 |
| 27 | 1 | 6 | 70% Ethanol | 27.1 |
| 28 | 1 | 0 | 100% Ethanol | 16.8 |
| 29 | 0 | 1 | 100% Ethanol | 19.4 |
| 30 | 1 | 1 | 100% Ethanol | 26.4 |
| 31 | 2 | 1 | 100% Ethanol | 24.1 |
| 32 | 4 | 1 | 100% Ethanol | 23.8 |
| 33 | 6 | 1 | 100% Ethanol | 21.0 |
| 34 | 1 | 2 | 100% Ethanol | 24.2 |
| 35 | 1 | 4 | 100% Ethanol | 22.9 |
| 36 | 1 | 6 | 100% Ethanol | 22.1 |

### [Example 2] Analysis of Nitric Oxide Production

In order to determine the effect of the Salvia miltiorrhiza or Paeonia lactiflora extract on nitric oxide (hereinafter referred to as NO) production during vascular aging caused by oxidative stress, HAoECs were treated H₂O₂ and then NO production was analyzed. Specifically, HAoECs were seeded in 96 plates at 5×10⁴ cells/well, incubated for 24 hours in an incubator at 37° C. under 5% CO₂. The cells were treated with 50 μg/ml of the extracts in Preparation Examples 1 to 36 and H₂O₂ and incubated for 24 hours. Subsequently, the total NO production was measured in supernatant isolated from the cells at absorbance 570 nm using a NO assay kit. NO recovery capacity was calculated using the following Equation 3.

NO recovery capacity (%)={(Absorbance of untreated group−Absorbance of extract-treated group)/(Absorbance of untreated group−Absorbance of H₂O₂-treated group)}×100 [Equation 3]

As a result, as shown in Table 4, the NO recovery capacity of the mixed extract was better than that of the single extract, the NO recovery capacity of 70% ethanol extract was the best among the extraction solvents, and in particular, Preparation Example 21 showed the highest NO recovery capacity of 39.7% compared to the NO reduction by H₂O₂.

TABLE 4

| Preparation | Salvia | Paeonia | Extraction | NO Recovery |
|---|---|---|---|---|
| Example | miltiorrhiza | lactiflora | solvent | capacity (%) |
| 1 | 1 | 0 | Water | 22.1 |
| 2 | 0 | 1 | Water | 18.5 |
| 3 | 1 | 1 | Water | 32.1 |
| 4 | 2 | 1 | Water | 27.5 |
| 5 | 4 | 1 | Water | 28.5 |
| 6 | 6 | 1 | Water | 27.4 |
| 7 | 1 | 2 | Water | 25.7 |
| 8 | 1 | 4 | Water | 24.7 |
| 9 | 1 | 6 | Water | 23.8 |
| 10 | 1 | 0 | 30% Ethanol | 22.5 |
| 11 | 0 | 1 | 30% Ethanol | 19.1 |
| 12 | 1 | 1 | 30% Ethanol | 33.5 |
| 13 | 2 | 1 | 30% Ethanol | 28.9 |
| 14 | 4 | 1 | 30% Ethanol | 27.5 |
| 15 | 6 | 1 | 30% Ethanol | 26.7 |
| 16 | 1 | 2 | 30% Ethanol | 26.5 |
| 17 | 1 | 4 | 30% Ethanol | 24.5 |
| 18 | 1 | 6 | 30% Ethanol | 24.5 |
| 19 | 1 | 0 | 70% Ethanol | 25.9 |
| 20 | 0 | 1 | 70% Ethanol | 20.1 |
| 21 | 1 | 1 | 70% Ethanol | 39.7 |
| 22 | 2 | 1 | 70% Ethanol | 32.5 |
| 23 | 4 | 1 | 70% Ethanol | 32.1 |
| 24 | 6 | 1 | 70% Ethanol | 30.2 |
| 25 | 1 | 2 | 70% Ethanol | 27.5 |
| 26 | 1 | 4 | 70% Ethanol | 27.4 |
| 27 | 1 | 6 | 70% Ethanol | 26.5 |
| 28 | 1 | 0 | 100% Ethanol | 25.5 |
| 29 | 0 | 1 | 100% Ethanol | 18.5 |
| 30 | 1 | 1 | 100% Ethanol | 34.2 |
| 31 | 2 | 1 | 100% Ethanol | 29.7 |
| 32 | 4 | 1 | 100% Ethanol | 28.5 |
| 33 | 6 | 1 | 100% Ethanol | 28.4 |
| 34 | 1 | 2 | 100% Ethanol | 26.4 |
| 35 | 1 | 4 | 100% Ethanol | 26.8 |
| 36 | 1 | 6 | 100% Ethanol | 26.9 |

### [Example 3] Analysis of Collagen Fiber Production

In order to determine the effect of the Salvia miltiorrhiza or Paeonia lactiflora extract on collagen fiber production during vascular aging caused by inflammatory response, HAoSMCs were treated with TNF-α to induce vascular aging by inflammatory response, and then collagen fiber production was analyzed. Specifically HAoSMCs were seeded in a 24 well plate at 5×10⁵ cells/well and then incubated for 24 hours in an incubator at 37° C. under 5% CO₂. The cells were treated with 50 μg/ml of the extracts in Preparation Examples 1 to 36 and TNF-α and incubated for 24 hours. the total collagen fiber production was measured in supernatant isolated from the cells at absorbance 450 nm using a collagen assay kit. The collagen fiber production inhibitory ability was calculated using the following Equation 4.

Collagen fiber production inhibitory ability (%)={(Absorbance of untreated group−Absorbance of extract-treated group)/(Absorbance of untreated group−Absorbance of TNF-α-treated group)}×100 [Equation 4]

As a result, as shown in Table 5, it was found that collagen fiber production increased in the TNF-α-treated group, while decreased in Preparation Examples 1 to 36. Specifically, the collagen fiber production inhibitory ability of the mixed extract was superior than that of the single extract, 70% ethanol extract was the best among the extraction solvents, and in particular, it was found that the Preparation Example 21 showed the highest inhibitory ability of 58.5%.

TABLE 5

| | | | | Collagen |
|---|---|---|---|---|
| | | | | fiber |
| | | | | production |
| | | | | inhibitory |
| Preparation | Salvia | Paeonia | Extraction | ability |
| Example | miltiorrhiza | lactiflora | solvent | (%) |
| 1 | 1 | 0 | Water | 14.8 |
| 2 | 0 | 1 | Water | 15.2 |
| 3 | 1 | 1 | Water | 30.2 |
| 4 | 2 | 1 | Water | 24.5 |
| 5 | 4 | 1 | Water | 22.2 |
| 6 | 6 | 1 | Water | 16.8 |
| 7 | 1 | 2 | Water | 23.7 |
| 8 | 1 | 4 | Water | 22.8 |
| 9 | 1 | 6 | Water | 17.5 |
| 10 | 1 | 0 | 30% Ethanol | 21.7 |
| 11 | 0 | 1 | 30% Ethanol | 23.5 |
| 12 | 1 | 1 | 30% Ethanol | 42.5 |
| 13 | 2 | 1 | 30% Ethanol | 38.5 |
| 14 | 4 | 1 | 30% Ethanol | 34.5 |
| 15 | 6 | 1 | 30% Ethanol | 32.1 |
| 16 | 1 | 2 | 30% Ethanol | 37.1 |
| 17 | 1 | 4 | 30% Ethanol | 33.5 |
| 18 | 1 | 6 | 30% Ethanol | 30.7 |
| 19 | 1 | 0 | 70% Ethanol | 28.5 |
| 20 | 0 | 1 | 70% Ethanol | 27.5 |
| 21 | 1 | 1 | 70% Ethanol | 58.5 |
| 22 | 2 | 1 | 70% Ethanol | 48.5 |
| 23 | 4 | 1 | 70% Ethanol | 38.5 |
| 24 | 6 | 1 | 70% Ethanol | 32.6 |
| 25 | 1 | 2 | 70% Ethanol | 43.2 |
| 26 | 1 | 4 | 70% Ethanol | 36.9 |
| 27 | 1 | 6 | 70% Ethanol | 30.8 |
| 28 | 1 | 0 | 100% Ethanol | 19.5 |
| 29 | 0 | 1 | 100% Ethanol | 21.5 |
| 30 | 1 | 1 | 100% Ethanol | 34.5 |
| 31 | 2 | 1 | 100% Ethanol | 29.4 |
| 32 | 4 | 1 | 100% Ethanol | 27.5 |
| 33 | 6 | 1 | 100% Ethanol | 22.8 |
| 34 | 1 | 2 | 100% Ethanol | 27.4 |
| 35 | 1 | 4 | 100% Ethanol | 25.9 |
| 36 | 1 | 6 | 100% Ethanol | 23.4 |

### [Example 4] Analysis of Vascular Aging Inhibitory Activity in Animal Models

In the above Examples 1 to 3, it was found that Example 21 was the best in cytoprotective ability against cytotoxicity, NO recovery capacity, and collagen fiber production inhibitory ability, and accordingly, in order to determine the effect of Preparation Example 21 on vascular health in an animal model, vascular aging-induced Sprague-Dawley Rat animal models induced by a high-cholesterol diet were used to analyze the vascular aging inhibitory activity of Example 21.

A 7-week-old male Sprague-Dawley Rat weighing 180 to 220 g was purchased from Koatech and all animal care and testing procedures were strictly carried out in accordance with the internationally recognized guidelines for the use of laboratory animals (IACUC). After receiving the laboratory animal, they were adapted to the new environment under constant temperature with 12/12 hours of a light/dark cycle and underwent an inspection and quarantine process through observation of external symptoms. For 7 days, they were given free intake of general solid feed and drinking water, and after an adaptation period, animals were randomly divided according to body weight. The experimental groups (G2 to G6) except the normal group (G1) were allowed to freely intake the high-cholesterol diet (1.25% cholesterol diet [D12336]) to induce vascular aging, and as shown in Table 6, the extract in Preparation Example 21 was administered orally every day for 12 weeks for each concentration (G4 to G6). The omega-3 treatment group (G3) which has an inhibitory effect on vascular aging was set as a positive control group.

The type and severity of general symptoms, including death, were observed and recorded once a day during the breeding period for experimental animals, and weight, feed, and drinking water intake; and blood pressure were measured once a week. One day before the end of the test, all living animals were fasted for one night. The next day, anesthesia was performed and then all groups were isolated the aortic blood vessels were removed and fixed in a 10% neutral buffered formalin solution.

The fixed tissues were prepared as a specimen for histopathological examination by undergoing general tissue treatment processes such as trimming, dehydration, paraffin embedding, and sectioning. Aortic vessel sections were stained with Hematoxylin & Eosin (H&E) or Masson's Trichrome, respectively, and aortic vessel thickness and aortic fibrosis were measured using an image analyzer (Zen 2.3 blue edition, Carl Zeiss, Germany).

TABLE 6

| | |
|---|---|
| Group 1 (G1) | Normal group |
| Group 2 (G2) | High cholesterol diet group (Vascular aging-induced group) |
| Group 3 (G3) | High cholesterol diet + Omega-3-administered group |
| Group 4 (G4) | High cholesterol diet + Low-concentration Preparation |
| | Example 21-administered group (400 mg × 1 time) |
| Group 5 (G5) | High cholesterol diet + Medium-concentration Preparation |
| | Example 21-administered group (800 mg × 1 time) |
| Group 6 (G6) | High cholesterol diet + High-concentration Preparation |
| | Example 21-administered group (600 mg × 2 times) |

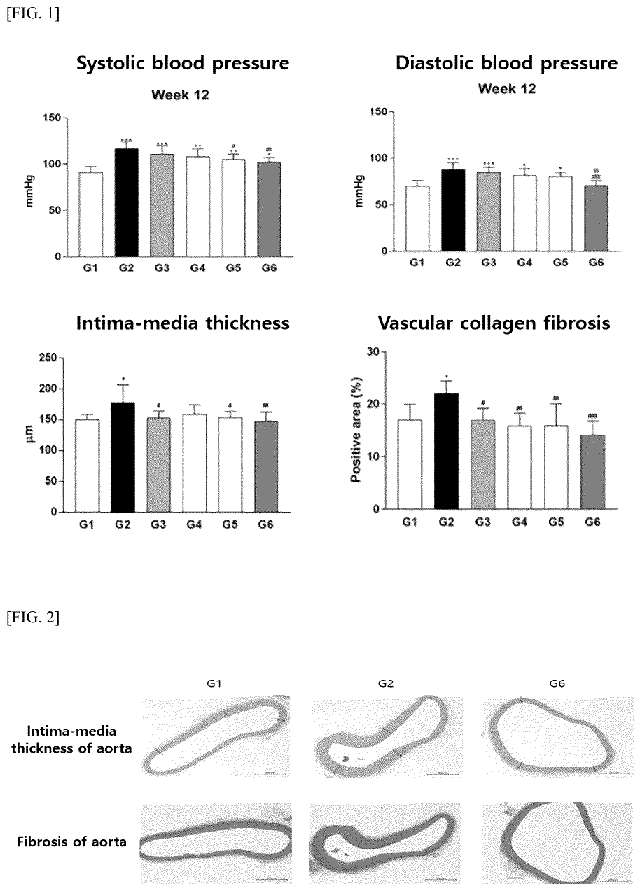

As a result, as shown in FIG. 1, it was found that both systolic and diastolic blood pressures were significantly reduced in a concentration dependent manner in groups treated with the extract in Preparation Example 21 at 12 weeks after administration of the extract. In addition, as shown in FIGS. 1 and 2, the intima-media thickness of the aorta was thicker in the vascular aging-induced group (G2) than the normal group (G1), while in the groups (G4 to G6) treated with extracts in Preparation Example 21, the increase in the intima-media thickness due to vascular aging was suppressed in a concentration dependent manner. In addition, it was found that collagen fiber deposition cause by vascular aging was also inhibited in a concentration dependent manner in groups (G4 to G6) treated with extracts of Preparation Example 21.

Having described a specific part of the contents of the present disclosure in detail, it is clear for a person skilled in the art that such a specific technique is only a preferred example embodiment, and the scope of the present disclosure is not limited thereby. That is, the substantive scope of the present disclosure is defined by the attached claims and their equivalents.

What is claimed is:

1. A method of preventing or treating diseases caused by vascular aging, comprising:
administering a pharmaceutical composition comprising: a Salvia miltiorrhiza extract; a Paeonia lactiflora extract; or a mixed extract of Salvia miltiorrhiza and Paeonia lactiflora as an active ingredient to a subject.

2. The method of claim 1, wherein the extract is extracted with water, a low-grade C1-C4 alcohol, or a mixed solvent thereof.

3. The method of claim 1, wherein the mixed extract is obtained by mixing the Salvia miltiorrhiza extract and the Paeonia lactiflora extract in a weight ratio of 1:0.1 to 10.

4. The method of claim 1, wherein the extract exhibits a cytoprotective effect against cytotoxicity induced by vascular aging.

5. The method of claim 1, wherein the extract promotes nitric oxide production.

6. The method of claim 1, wherein the extract inhibits collagen fiber production.

7. The method of claim 1, wherein the disease is one or more selected from the group consisting of atherosclerosis, hyperlipidemia, hypertension, angina, myocardial infarction, stroke, cardiac arrhythmia, heart failure, endocarditis, ischemic cerebrovascular disease, and ischemic heart disease.

8. A method of preventing or ameliorating diseases caused by vascular aging, comprising:
administering a health functional food composition comprising a Salvia miltiorrhiza extract; a Paeonia lactiflora extract; or a mixed extract of Salvia miltiorrhiza and Paeonia lactiflora as an active ingredient to a subject.

9. A method of preventing or ameliorating symptoms caused by vascular aging, comprising:
administering a food composition comprising a Salvia miltiorrhiza extract; a Paeonia lactiflora extract; or a mixed extract of Salvia miltiorrhiza and Paeonia lactiflora as an active ingredient to a subject.

10. The method of claim 9, wherein the symptom induced by vascular aging is one or more selected from the group consisting of a decrease in the elasticity of blood vessels, an increase in the stiffness of blood vessels, expansion of an inner diameter of blood vessels, an increase in the thickness of the vascular wall of blood vessels, degeneration of the intima-media membrane of blood vessels, a decrease in dilatability of blood vessels, insufficiency of endothelial cells of blood vessels, an increase in reactive oxygen species (ROS) of blood vessels, an inflammatory response in blood vessels, proliferation of smooth muscle cells in the subendothelial space of blood vessels, a decrease in blood flow rate in blood vessels, and an increase in blood pressure.
